(19) United States
(12) Patent Application Publication
(10) Pub. No.: US 2026/0183444 A1
(43) Pub. Date: Jul. 2, 2026

(54) CYCLONIC ULTRAVIOLET AIR PURIFICATION CHAMBER

(71) Applicant: CANON VIRGINIA, INC., Newport News, VA (US)

(72) Inventors: Maxwell Hensley, Williamsburg, VA (US); Ugur Erturun, Norfolk, VA (US); Raymond Dawson, Hampton Roads, VA (US); Ralph McCann, Williamsburg, VA (US)

(21) Appl. No.: 19/549,780

(22) Filed: Feb. 25, 2026

Related U.S. Application Data
(63) Continuation of application No. PCT/US2024/044194, filed on Aug. 28, 2024.
(60) Provisional application No. 63/579,786, filed on Aug. 30, 2023.

(51) Int. Cl. A61L 9/20; B01D 46/00; B01D 53/00; B01D 53/88

(52) U.S. Cl. CPC A61L 9/205; B01D 46/0028; B01D 46/0043; B01D 46/0045; B01D 53/007; B01D 53/885; A61L 2209/12; A61L 2209/14; B01D 2255/802; B01D 2255/915; B01D 2259/804; B01D 2273/30; B01D 2279/65

(57) ABSTRACT

Disclosed are methods and devices for air purification using photocatalytic oxidation that include an air purification device that includes an inlet; a first chamber including at least one first emitter and at least one filter media, with the at least one first emitter configured to irradiate at least a surface of the at least one filter media; a second chamber configured to receive air from the first chamber, with the second chamber including at least one second emitter; at least one plate provided in the second chamber; and an exhaust formed on the second side of the second chamber, with the at least one second emitter being configured to irradiate at least a portion of the at least one plate, and with the second chamber being configured to induce cyclonic air flow therein.

### CROSS-REFERENCE TO RELATED APPLICATION(S)

The present application is a Continuation of International Patent Application No. PCT/US2024/044194, filed Aug. 28, 2024, which claims priority to U.S. Provisional Patent Application No. 63/579,786, filed with the U.S. Patent and Trademark Office on Aug. 30, 2023, both of which are incorporated herein by reference.

### BACKGROUND

### Field of the Disclosure

The present disclosure relates to air purification using photocatalytic oxidation.

### Description of Related Art

Airborne pollutants may include dust, allergens, and micro-organisms adverse to the health of persons breathing the air. Thus, air purifiers are used to remove airborne pollutants.

Air purifiers generally include a housing that provides an airflow path from an air inlet and air outlet. The airflow path may traverse an air filtering system, driven by a fan that moves air through the airflow path between the air inlet and the air outlet.

Categories of ultraviolet (UV) light wavelengths that are harmful to microorganisms include UV-A, UV-B, and UV-C. UV-A wavelengths are generally between 400 and 320 nanometers. UV-B wavelengths are generally between 320 nanometers and 290 nanometers. UV-C wavelengths are generally between 290 nanometers and 100 nanometers.

Treatment of surfaces with UV-C light reduces the levels of bacteria and funguses, and provides an effective, chemical-free method for infection prevention. However, UV-C light may be hazardous to skin and eyes.

U.S. Pub. 2023/0119976 discusses intelligent sensors to monitor airflow. U.S. Pat. No. 10,307,504 discusses a shelf securable disinfecting apparatus that includes a UV light source coupled to the shelf, with the UV light being configured to sanitize air and surfaces in close proximity to the UV light source; as well as motion detector, output of which causes energization or de-energization of the UV light source, to selectively disinfect or sterilize air and/or surfaces.

U.S. Pat. No. 10,512,879 discusses a system and a method for reducing hazardous gases, including indoor priority hazard gases (PHGs), through one or more photocatalysts in a filter system, with a microstructure of a photocatalytic filter formed using biological systems as a template for the photocatalysts to be deposited thereon.

U.S. Pat. No. 11,103,611 discusses a system for reducing infection by controlling and/or reducing the level of contaminants. The system including an inlet passage receiving inlet air from the environment; a humidifier input receiving water vapor from a humidification device; a mixer where the inlet air and water vapor are mixed to form an air/vapor mixture; a controller and humidity sensor; and a treatment chamber wherein the air/vapor mixture is subject to a photocatalytic oxidation treatment, the treatment chamber including one or more ultraviolet light sources and including photocatalytic material configured to receive ultraviolet light emitted from the one or more ultraviolet light sources, which may be provided by one or more UV light emitting light bulbs or light emitting diodes (LEDs).

U.S. Pat. No. 10,092,672 discusses an air filtration media for use with a heating ventilation and air condition (HVAC) system, with the filtration media including an air filter media layer having a first and second side, a photocatalytic oxidation (PCO) media layer having a first and second side, and a barrier layer positioned between the second side of the air filter media layer and the first side of the PCO media layer. The air filter media layer, barrier layer, and PCO layer are pleated together and enclosed within a frame for placement adjacent a light source within a plenum of the HVAC system.

U.S. Pat. No. 8,529,831 discusses an air purification system based on in-situ photocatalytic oxidation and ozonation includes a single multi-functional Ti02-based coating having photocatalytic activity for oxidation in the presence of a sufficient ozone supply and UV irradiation to synergistically oxidize gaseous pollutants at ambient conditions, and a method for removing gaseous pollutants using ozone and UV irradiation to simultaneously activate the photocatalytic oxidation in the presence of the Ti02-based coating to remove up to about 84% of the gaseous pollutants within five minutes.

U.S. Pub. 2021/228,762 discusses an air purification device having a housing for holding a PCO unit and a fan assembly, and optionally including a filter compartment for holding a filter such as a HEPA filter to provide effective purification and sanitation of air in a targeted indoor environment, and in particular in a medical environment such as a hospital.

U.S. Pub. 2021/393,846 discloses an apparatus for reducing infection by controlling and/or reducing the level of contaminants, which include pathogens, allergens and/or odor-causing agents including VOCs, with photocatalytic oxidation being performed in a high humidity environment so that hydrogen peroxide molecules are readily produced.

The disclosure of each of the above patents and patent publications is incorporated herein by reference,

However, conventional systems provide high airflow volume through one portion of an illuminated area without sufficient dwell time while allowing smaller volumes to other illuminated areas. Thus, conventional system fail to minimize UV-C light leakage/emission and also fail to evenly distribute airflow through a homogeneous illumination field, resulting in output of untreated air and/or reduced energy efficiency.

### SUMMARY

To overcome the shortcomings of conventional systems, the present application overcomes shortcomings of conventional devices and methods by providing methods and devices that maximize residence time of air particles in a vortex window that is proximate to an exhaust.

An aspect of the present disclosure provides an air purification device that includes an inlet; a first chamber including at least one first emitter and at least one filter media, with the at least one first emitter configured to irradiate at least a surface of the at least one filter media; a second chamber configured to receive air from the first chamber, with the second chamber including at least one second emitter; and at least one plate provided in the second chamber, with the at least one second emitter being configured to irradiate at least a portion of the at least one plate, and with the second chamber being configured to induce cyclonic air flow therein.

Another aspect of the present disclosure provides an air purification device that includes an inlet; a first chamber; a second chamber configured to receive air from the first chamber; and at least one plate provided in the second chamber, with the second chamber including at least one emitter configured to irradiate at least a portion of the at least one plate, and with the second chamber being configured to induce cyclonic air flow therein.

A further aspect of the present disclosure provides an air purification device that includes an inlet, a chamber configured to induce cyclonic flow of air from the inlet, and at least one plate provided in the chamber, with the chamber including at least one emitter configured to irradiate at least a portion of the at least one plate.

Another aspect of the present disclosure provides a method of purifying air by an air purification device that includes an inlet; a first chamber; a second chamber including at least one plate and at least one emitter; and an exhaust, with the method including flowing air from the inlet through the first chamber and the second chamber to the exhaust; and irradiating, by the at least one emitter, at least a portion of the at least one plate, with the second chamber being configured to induce cyclonic air flow therein.

### BRIEF DESCRIPTION OF THE DRAWINGS

The accompanying drawings, which are incorporated herein and form part of the specification, illustrate various embodiments, objects, features, and advantages of the present disclosure.

FIG. 1 illustrates a linear air purification device according to an embodiment.

FIG. 2 illustrates components of a cyclonic air purification device according to an embodiment.

FIG. 3 is an exploded view illustrating components of the cyclonic air purification device according to an embodiment.

FIG. 4 is a profile view illustrating components of the substantially linear first chamber according to an embodiment.

FIG. 5 illustrates results from computation fluid dynamic (CFD) simulation and particle tracing study for optimization according to an embodiment.

FIG. 6 illustrates results from CFD simulation to optimize height of the catalyst plate in the cyclonic chamber according to an embodiment.

FIG. 7 illustrates results from a ray tracing simulation for light leakage with only a catalyst plate according to an embodiment.

FIG. 8 illustrates results from a ray tracing simulation for light leakage with a photocatalyst coating applied to the catalyst plate and the outlet tube according to an embodiment.

FIG. 9 illustrates ray tracing simulation results, depicting irradiance at upper and lower surfaces of the catalyst plate according to an embodiment.

FIG. 10 illustrates results from CFD simulation of a tapered outlet cap according to an embodiment.

FIG. 11 illustrates light leakage from the tapered outlet cap according to an embodiment.

FIG. 12 is a table summarizing experimental test result data according to embodiments.

FIG. 13A illustrates a circular arrangement of the plurality of second emitters according to an embodiment.

FIG. 13B illustrates a strip of second emitters in a circular arrangement according to an embodiment.

FIG. 13C illustrates vortexes formed by a plurality of perforations according to an embodiment.

FIG. 13D illustrates an internal fan according to an embodiment.

FIG. 14A is a side profile illustrating stacking of multiple catalyst plates according to an embodiment.

FIG. 14B is a side profile illustrating stacking of multiple catalyst plates according to another embodiment.

FIG. 15A illustrates an outlet tube and FIGS. 15B to 15D illustrate outlet caps according to embodiments.

FIGS. 16A-G illustrate arrangements of the plate according to embodiments.

Throughout the figures, the same reference numerals, and characters, unless otherwise stated, are used to denote like features, elements, components, or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENT(S)

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

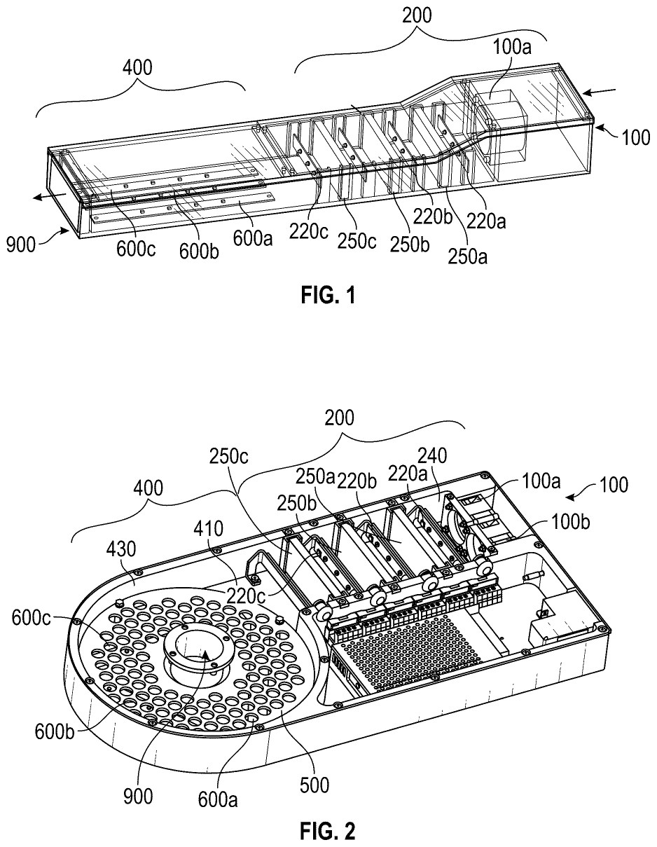

FIG. 1 illustrates a linear air purification device according to an embodiment.

The device of FIG. 1 is configured to linearly convey air sequentially through a UV-A and UV-C purification chambers. As illustrated in FIG. 1, an inlet fan 100a is positioned an or adjacent to inlet 100 to a first chamber 200. After flowing through the first chamber 200, the air flows through a second chamber 400 to an exhaust 900. The first chamber 200 includes at least one first emitter 220a, 220b, 220c and at least one filter media 250a, 250b, 250c, with an output of the at least one first emitter 220a, 220b, 220c positioned to emit radiation to irradiate at least a surface of the at least one filter media 250a, 250b, 250c. At least a portion of the at least one filter media 250a, 250b, 250c opposite the at least one first emitter 220a, 220b, 220c may be coated with a photocatalyst that is activated by at least one of UV-A or UV-C radiation, to break down contaminants by photocatalytic oxidation. The at least one first emitter 220a, 220b, 220c may be a UV-A emitter, and the at least one second emitter 600a, 600b, 600c may be a UV-C emitter.

The second chamber 400 receives air from the first chamber 200, and the second chamber 400 includes at least one second emitter 600a, 600b, 600c, an output of which irradiates at least a portion of an interior of the second chamber 400. At least a portion of the interior of the second chamber 400 opposite the at least one second emitter 600a, 600b, 600c may be coated with a photocatalyst that is activated by at least one of UV-A or UV-C radiation, to cause photocatalytic oxidation of at least one of a particle and a gaseous molecule, and break down contaminants by photocatalytic oxidation.

FIG. 2 illustrates components of a cyclonic air purification device according to an embodiment.

As illustrated in FIG. 2, the air purification device 1000 may include an inlet 100, a first chamber 200, and a second chamber 400. A plurality of fans 100a, 100b may be provided at an inlet of the first chamber 200. The plurality of fans 100a, 100b may be arranged with an airflow channel aspect ratio to provide homogenous airflow through the first chamber 200.

The first chamber 200 may include at least one first emitter 220a, 220b, 220c and at least one filter media 250a, 250b, 250c. The at least one filter media 250a, 250b, 250c includes or is coated with a photocatalyst that is activated by at least one of UV-A or UV-C radiation, to break down contaminants by photocatalytic oxidation. An output of the at least one first emitter 220a, 220b, 220c may be configured to irradiate a respective at least one surface of the at least one filter media 250a, 250b, 250c with at least one of UV-A or UV-C radiation, to break down contaminants by the photocatalytic oxidation. At least one hole may be provided through one or more of the at least one first emitter 220a, 220b, 220c to maintain air flow through the first chamber 200.

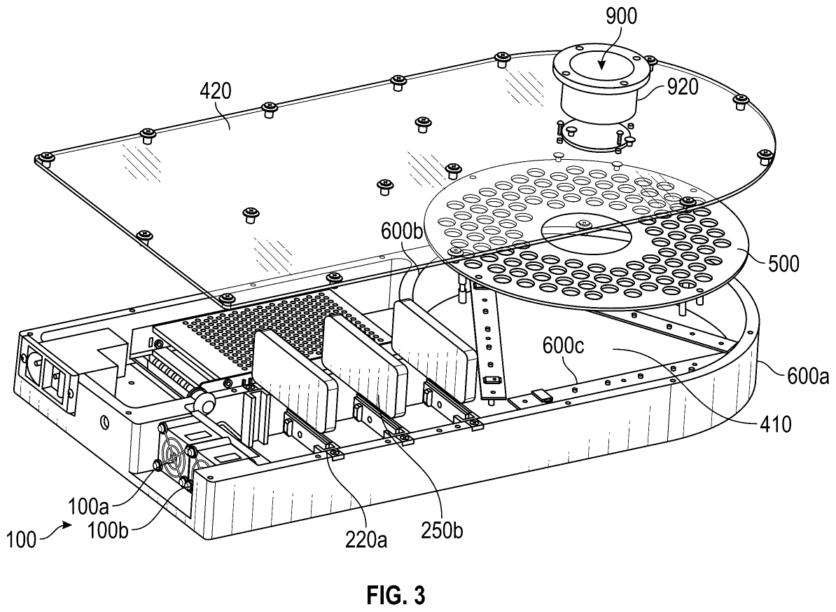

The second chamber 400, which receives air from the first chamber 200, may include a cyclonic chamber that is bounded by a first side 410 and a second side 420 (FIG. 3) substantially opposite the first side 410, with a substantially circular wall 430 connecting the first side 410 and the second side 420. The second chamber 400 may include at least one second emitter 600, an output of which may be positioned to irradiate a photocatalyst that is provided on or in at least one plate 500 that may be provided in the second chamber 400.

Air exiting the second chamber 400 flows through an exhaust 900 that may be formed substantially in the center of the second side 420 of the second chamber 400. The at least one plate 500 may include a hole 560 located substantially at a center of the at least one plate 500, substantially aligned with the exhaust 900. Thus, air flowing through the second chamber 400 may pass through the hole 560 of the at least one plate 500 before exiting through the exhaust 900. Thus, air enters the second chamber 400 in a direction that is orthogonal to a direction of air exiting through an exhaust 900 of the second chamber.

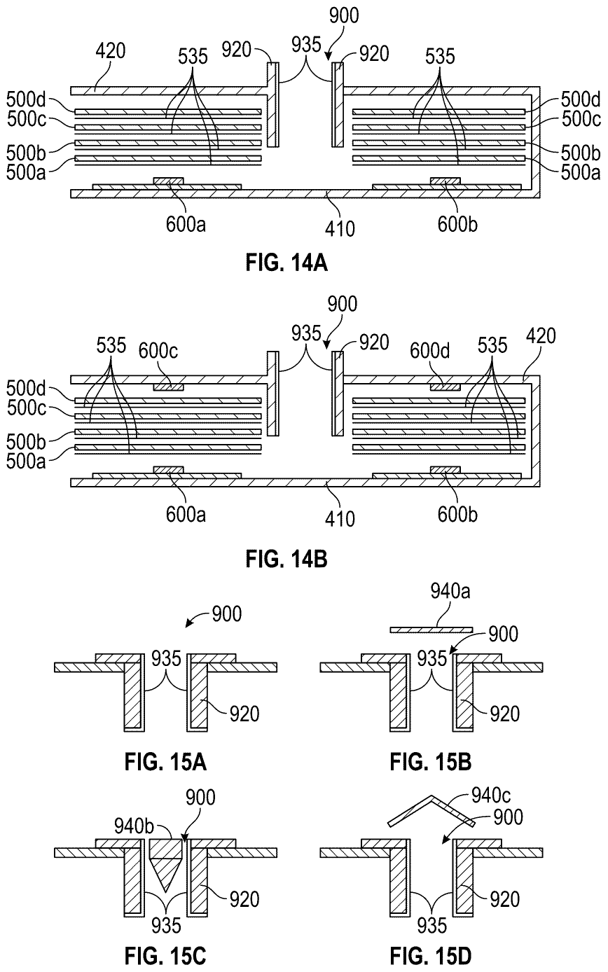

FIG. 3 is an exploded view illustrating components of the cyclonic air purification device according to an embodiment. As illustrate in FIG. 3, a configuration with the at least one second emitter being provided as three separate strips 600a, 600b, 600c, with each strip arranged to correspond to a shape of the at least one plate 500. A first side 410, i.e., a floor, and a second side 420, i.e., a ceiling, of the second chamber 400 are provided, with an outlet tube 920 that may extend through a hole 560 positioned substantially in a center of the at least one plate 500. Additional views of the outlet tube 920 extending through the hole 560 in the at least one plate 500 are provided in FIGS. 14A and 14B.

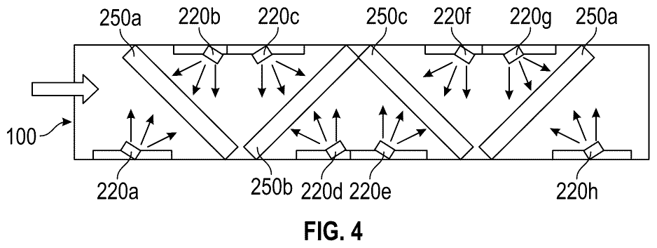

FIG. 4 is a profile view illustrating components of the substantially linear first chamber according to an embodiment. As illustrated in FIG. 4, a plurality of filter media 250a, 250b, 250c and a plurality of first emitters 220a, 220b, 220c are provided in the first chamber 200. Each filter media 250a, 250b, 250c is positioned with at least one opposite side thereof offset from a substantially perpendicular orientation, relative to a longitudinal wall of the first chamber 200; i.e., offset at a non-orthogonal angle to an inner surface of the longitudinal wall of the first chamber 200. Each emitter of the plurality of first emitters 220a, 220b, 220c is fixed to a longitudinal wall at a position along a length of the first chamber 200 to provide illumination of a surface of a respective side of a media of the plurality of filter media 250a, 250b, 250c. The angle of offset from the inner surface of the longitudinal wall of the first chamber 200 may vary from approximately ten degrees to approximately ninety degrees, primarily based a positioning of the UV light emitter and a respective angle of incidence upon a respective surface of the respective side of a media of the plurality of filter media 250a, 250b, 250c.

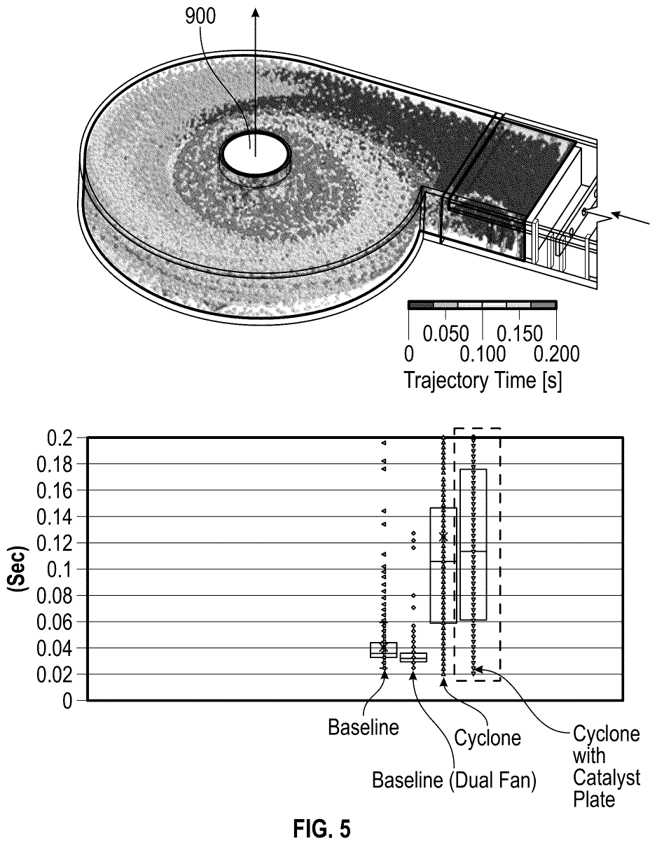

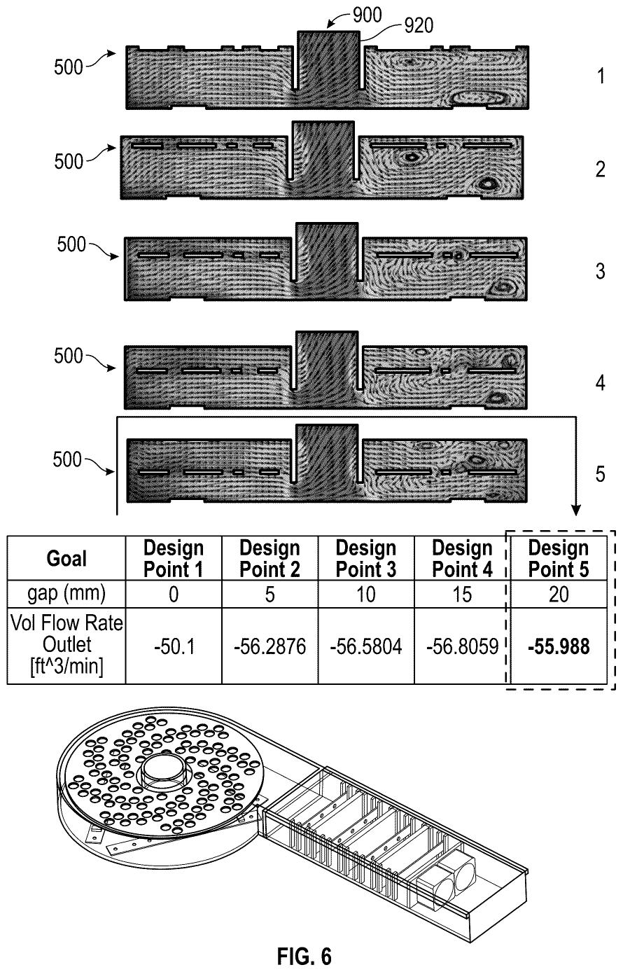

FIG. 5 illustrates results from CFD simulation and particle tracing study for optimization according to an embodiment. FIG. 5 provides a comparison of relative particle residence time between various chamber arrangements. Specifically, FIG. 5 compares a baseline linear arrangement, e.g., as illustrated in FIG. 1; a baseline linear dual fan arrangement; a cyclonic arrangement, e.g. FIG. 2, and a cyclonic arrangement combined with a catalyst plate, e.g. FIG. 6. As illustrated in FIG. 5, the cyclone arrangements provide improved particle residence time.

FIG. 6 illustrates results from CFD simulation to optimize height of the catalyst plate in the cyclonic chamber according to an embodiment. Five different heights of the catalyst plate 500 are illustrated on the left side of FIG. 6, ranging from adjacent to the second side 420 to adjacent to a lower end of outlet tube 920 of the exhaust 900. As shown on the right side of FIG. 6, a lowest position of plate 500, i.e., closest to the lower end of the outlet tube 920, optimizes flow rate, reduces airflow dead zones, more evenly moves contaminants through a homogeneous illumination field, and maximizes efficiency. Such increase of residence time of air within the chamber provides increases irradiance to improve efficiency of contaminant breakdown. Thus, an increased volumetric flow rate is obtained, allowing a greater volume of air to be conveyed through the system for a given period of time, allowing fewer units to be used to provide reduced contaminant levels for a given volume of space. In contrast, conventional systems rely on increasing airflow volume through one portion of the illuminated area, with a small volume, i.e., a non-progressing dead zone volume, existing in another illuminated area.

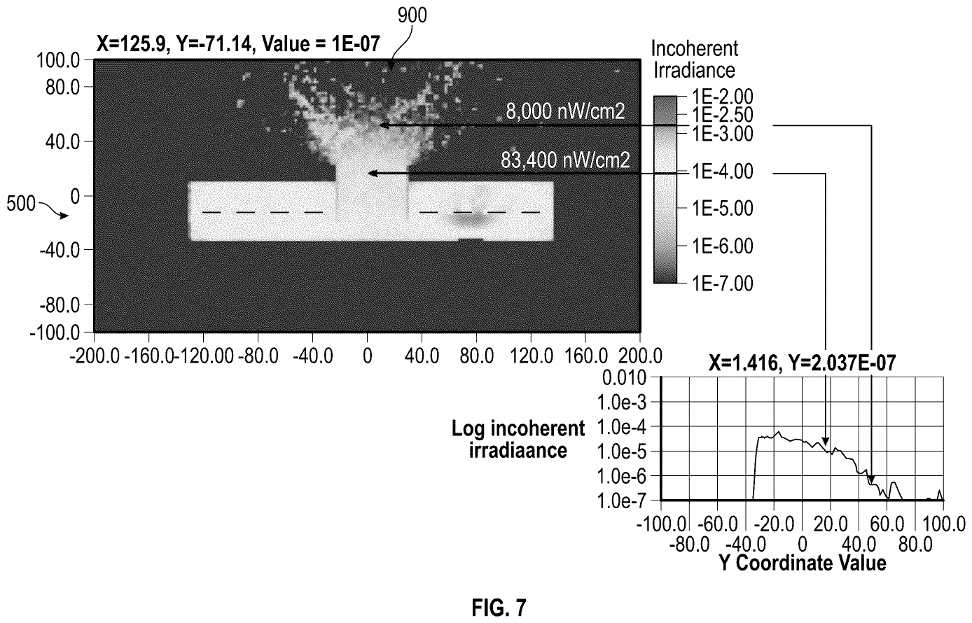

FIG. 7 illustrates results from a ray tracing simulation for light leakage/emission with only a catalyst plate according to an embodiment. With respect to the CFD and particle tracing studies, the cyclonic second chamber 200 and catalyst plate 500 provide approximately a ten times improvement in particle residence while also providing up to 55 CFM over a target value of 45 CFM minimum, with physical testing measuring a throughput of 65 CFM.

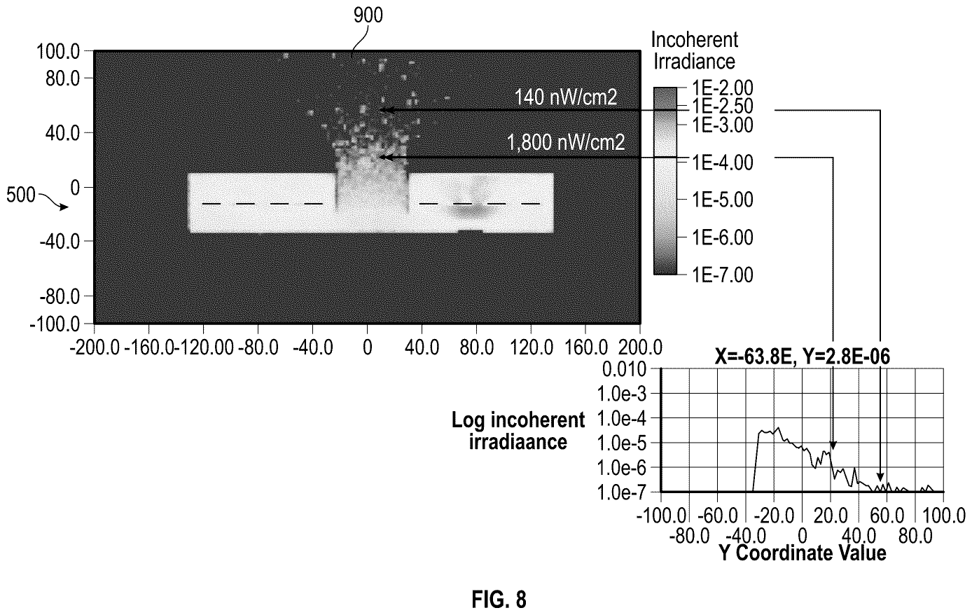

FIG. 8 illustrates results from a ray tracing simulation for light leakage/emission with a photocatalyst coating applied to the catalyst plate (FIGS. 14A, 14B) and the outlet tube (FIGS. 15A-15D) according to an embodiment. For UV-C light leakage, guidelines of the International Commission on Non-Ionizing Radiation Protection (ICNIRP) provide a target irradiance dosage limit of a maximum human biologically efficient radiant exposure of the eye and skin to UVR within a nominal eight hour period is 30 J/m² effective, roughly equating to a maximum irradiance of 100 nW/cm².

Comparison of FIG. 7 to FIG. 8 shows improved incoherent irradiance is obtained by application of coatings applied to the catalyst plate and the outlet tube according to an embodiment. At least a part of the outlet tube 920 may be coated with a photocatalyst that is configured to be activated by illumination from the at least one second emitter 600. Also, at least a part of a surface of the plate 500 may be coated with a photocatalyst that is configured to be activated by illumination. Coating the catalyst plate 500 and the outlet tube 920 reduced light leakage by providing approx. 140 nW/cm² at the target distance. Physical testing of the device measured 160 nW/cm², approx. 15% deviation, likely from slight mismatch of absorption and scattering dynamics, but still within the target range.

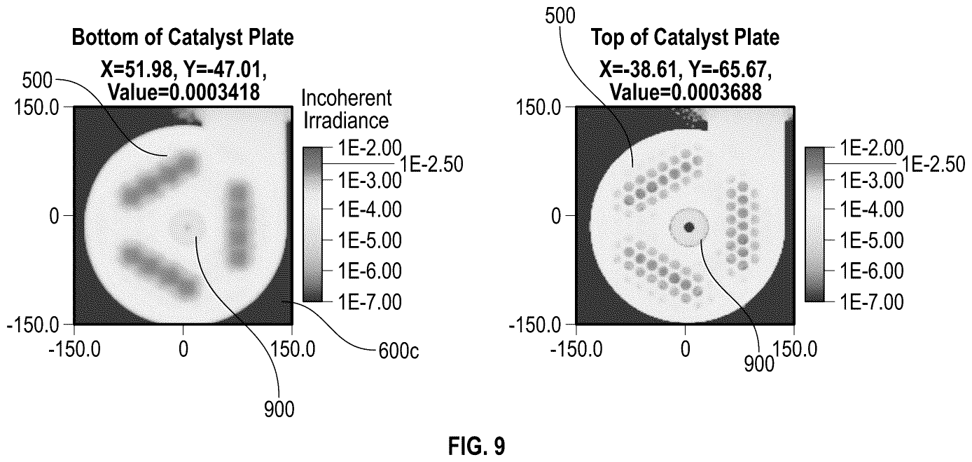

FIG. 9 illustrates ray tracing simulation results, depicting irradiance at upper and lower surfaces of the catalyst plate according to an embodiment. The left side of FIG. 9 provides results of the ray tracing simulation for the bottom of plate 500. The right side of FIG. 9 provides results of the ray tracing simulation for the top of plate 500. Perforations in the plate 500 allow air to better mix and to be conveyed through the plate 500. The perforations also allow UV light to pass through the plate 500, increasing reflectivity pathways for UV-C radiation to better reflect off the ceiling 420 as well as the walls of the second chamber 400, for re-absorption on an upper side of the plate 500, thus increasing the total irradiated area within the second chamber 400. As described herein, patterns of perforations may be further optimized based on LED placement and chamber geometry. A center of plate 500 is preferably positioned at the center of the second chamber 400, to aid improve flow conditions by providing pathways for flow separation and improved mixing and to also provide additional surface area for photocatalyst coating and irradiation.

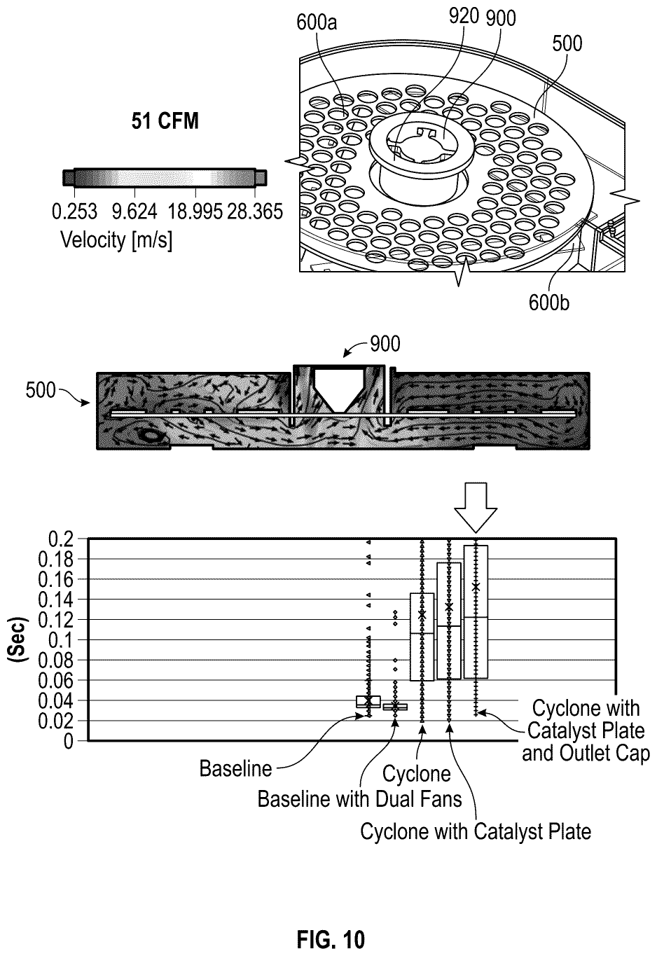

FIG. 10 illustrates results from CFD simulation of a tapered outlet cap according to an embodiment. FIG. 10 provides a comparison of a baseline linear arrangement, e.g., as illustrated in FIG. 1; a baseline linear dual fan arrangement; a cyclonic arrangement, e.g. FIG. 2, a cyclonic arrangement combined with a catalyst plate, e.g. FIG. 6; and a cyclonic arrangement combined with a catalyst plate, e.g. FIG. 15. As illustrated in FIG. 10, the tapered outlet cap reduces flow rate, improves particle residence time, and also lowers light leakage/emission, thereby efficiently containing light as UV-C irradiation to the second chamber 400. Minimizing light leakage/emission from the air purification device provides a safer environment for operators/individuals near the device.

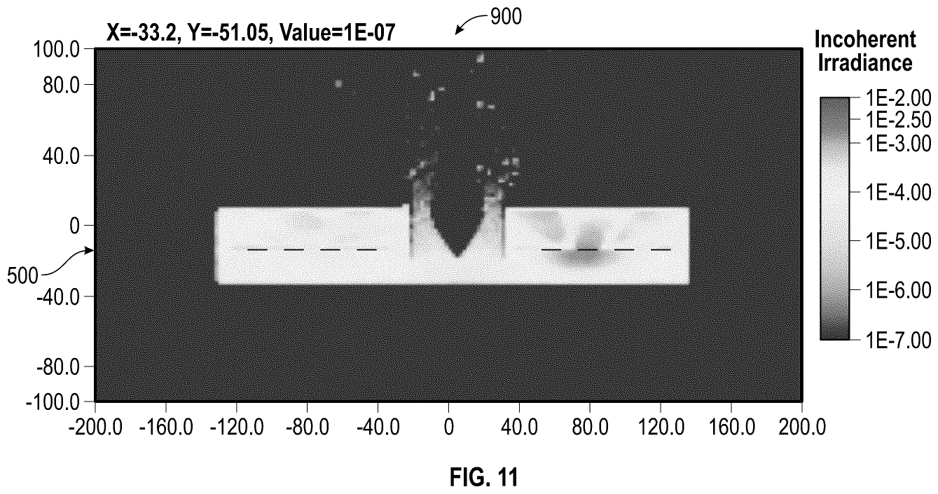

FIG. 11 illustrates light leakage/emission from the tapered outlet cap according to an embodiment.

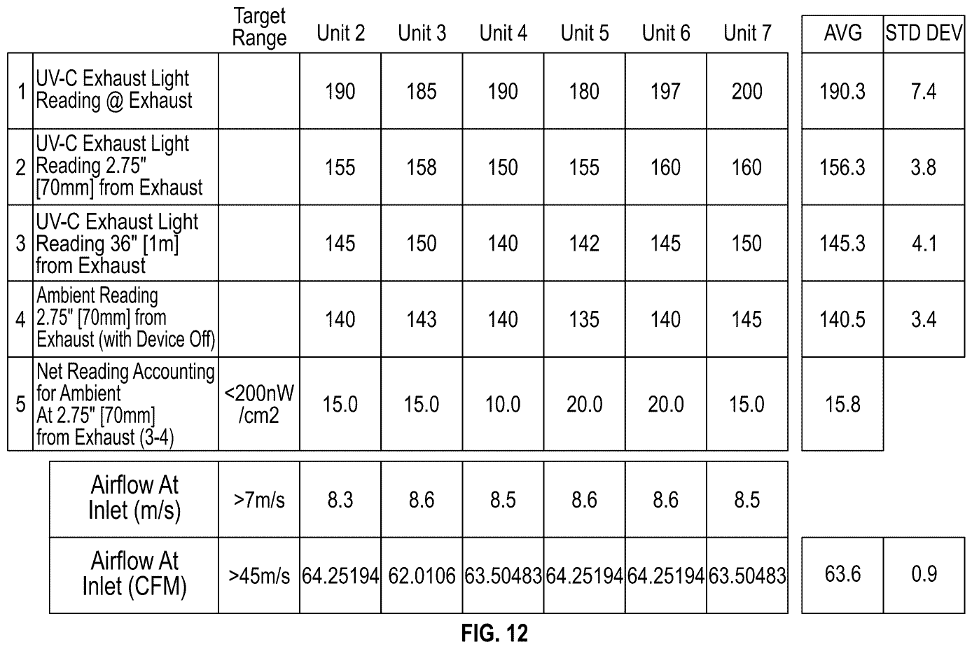

FIG. 12 is a table summarizing experimental test result data according to embodiments. FIG. 12 summarizes UV-C exhaust light, measured at varied distances outside the exhaust for different units.

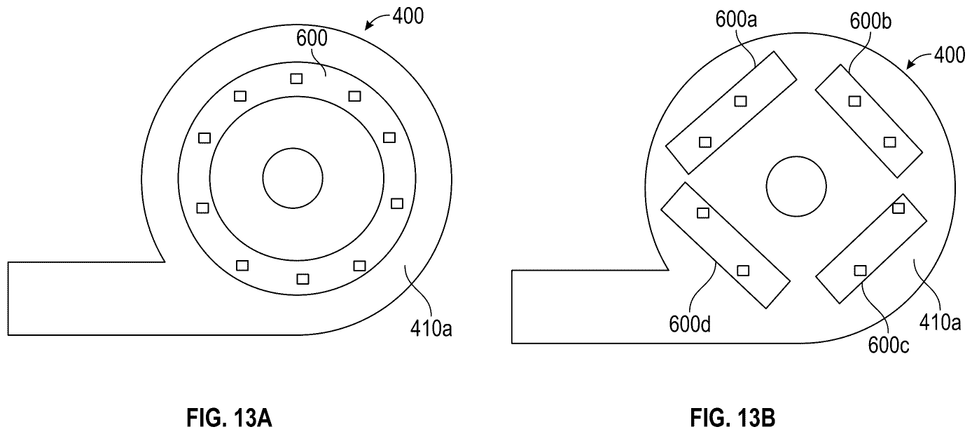

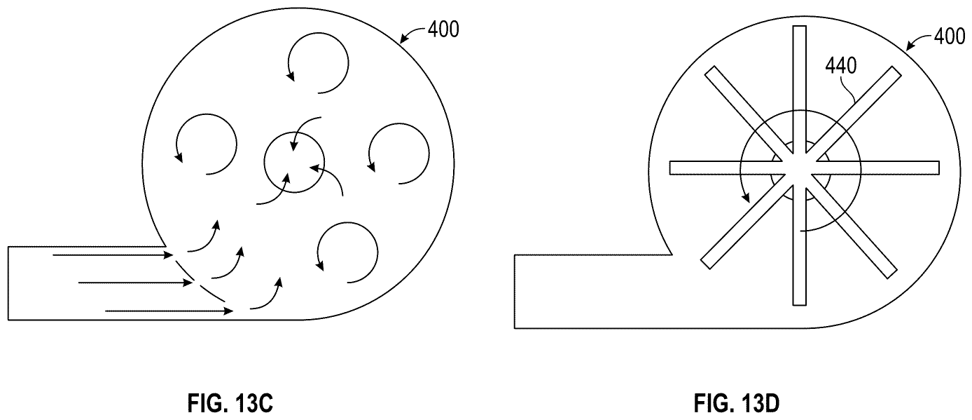

FIG. 13A illustrates a circular arrangement of the plurality of second emitters according to an embodiment. As illustrated in FIG. 13A, the plurality of second emitters 600 may be LEDs that are arranged on a single ring-shaped printed circuit board (PCB).

FIG. 13B illustrates an arrangement of strips of second emitters according to an embodiment. As illustrated in FIG. 13B, the plurality of second emitters 600 are provided on multiple PCB, with the PCB provided in a circular arrangement that corresponds to a shape of the at least one plate 500. Alternatively, individual LEDs modules may be mounted directly to the first side 410 of the second chamber 400 in the shape of a ring or other predetermined shape, without a PCB.

FIG. 13C illustrates vortexes formed by a plurality of perforations according to an embodiment. The plurality of air vortexes illustrated in FIG. 13C corresponds to patterns of perforations and/or protuberances provided on a surface of the plate 500.

FIG. 13D illustrates an internal fan according to an embodiment. When operated, the internal fan 440, i.e., impellor, circulates and forces air out of the exhaust 900, thus drawing air into the second chamber 400 from the first chamber 200. The internal fan 440 may be coated with photocatalyst material. The internal fan 440 may replace or be used in conjunction with the at least one inlet fan 100a, 100b, and/or a fan provided at the exhaust 900.

FIG. 14A is a side profile illustrating stacking of multiple catalyst plates according to an embodiment. FIG. 14B is a side profile illustrating stacking of multiple catalyst plates according to another embodiment. As illustrated in FIGS. 14A and 14B, a plurality of plates 500a, 500b, 500c, 500d are provided. FIG. 14A illustrates an embodiment with second emitters 600a and 600b are provided on only side of the plurality of plates 500a, 500b, 500c, 500d. FIG. 14B illustrates an embodiment with second emitters 600a and 600b provided on a lower side and second emitters 600c and 600d provided on an upper side of the plurality of plates 500a, 500b, 500c, 500d. Alternatively, second emitters 600a may be provided between each plate of the plurality of plates 500a, 500b, 500c, 500d. The plurality of plates 500a, 500b, 500c, 500d may be vertically stacked with separations therebetween, and with respective holes of each of the plurality of plates 500a, 500b, 500c, 500d aligned with the exhaust 900. As illustrated in FIGS. 14A and 14B, at least plate 500a may be positioned closer to the first side 410 of the second chamber 400 than a proximal end of the outlet tube 920.

As illustrated in FIGS. 14A and 14B, a photocatalyst coating 535 is provided on at least one surface of the plate 500 and a photocatalyst coating 935 is applied on an interior of the outlet tube to break down contaminants by photocatalytic oxidation and to reduce light leakage/emission.

FIG. 15A illustrates an outlet tube according to an embodiment. FIGS. 15B to 15D illustrate outlet caps according to embodiments. FIG. 15A is a side view illustrating the outlet tube 920 in the exhaust 900 without a cap. FIG. 15B illustrates an outlet cap 940a configured as a flat cover positioned on a distal end of the outlet tube 920. FIG. 15C illustrates a conical outlet cap 940b positioned on a distal end of the outlet tube 920. FIG. 15D illustrates a conical outlet cap 940c positioned on a distal end of the outlet tube 920. As shown in FIGS. 15A to 15D, a photocatalyst coating 935 may be applied on an inner circumference of the outlet tube 920, as well as on a surface of the outlet cap 940a, 940b, and 940c. Use of outlet cap 940a, 940b and/or 940c and application of the photocatalyst coating 935 on the inner circumference of the outlet tube 920 and/or on a surface of the outlet cap 940a, 940b, and 940c minimized UV-C light leakage. The outlet cap 940c may be hollow, i.e., without a bottom surface, as illustrated in FIG. 15D, or may be triangular shaped with a flat bottom.

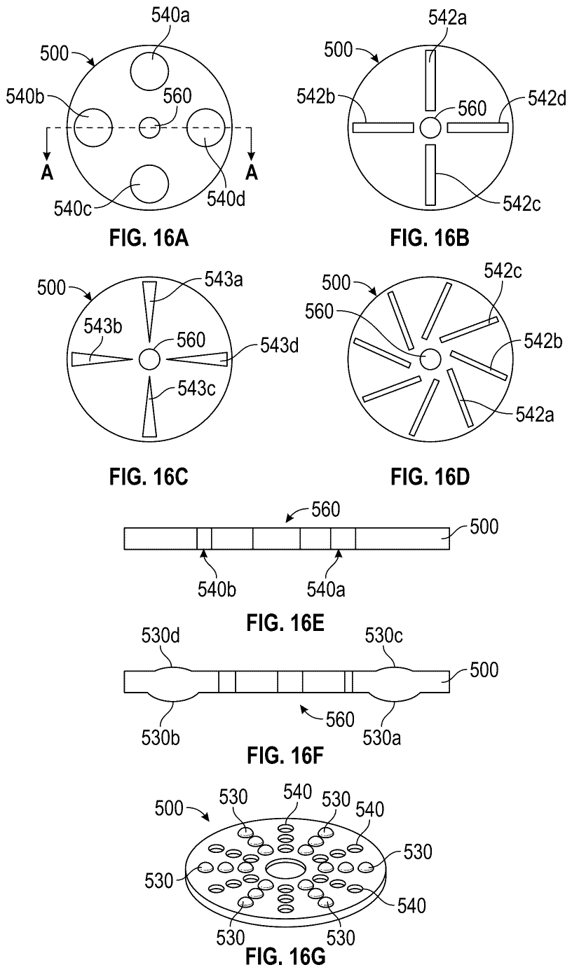

FIGS. 16A-G illustrate arrangements of the plate 500 according to embodiments.

FIG. 16A is a plan view of a plate 500 with a plurality of perforations 540a-540d according to an embodiment. The plurality of perforations 540a-540d extend into a surface of the plate 500 and one or more of the plurality of perforations 540a-540d may extend through the entire depth to create one or more holes in the plate 500. The plurality of perforations 540a-540d may encircle hole 560.

FIG. 16B is a plan view of a plate 500 with a plurality of slots 542a-542d according to an embodiment. The plurality of slots 542a-542d extend into a surface of the plate 500 and one or more of the plurality of slots 542a-542d may extend through the entire depth of the plate 500. The plurality of slots 542a-542d may encircle hole 560.

FIG. 16C is a plan view of a plate 500 with a plurality of triangular cutouts 544a-544d according to an embodiment. The plurality of triangular cutouts 544a-544d may extend into a surface of the plate 500 and one or more of the plurality of triangular cutouts 544a-544d may extend through the entire depth of the plate 500. The plurality of triangular cutouts 544a-544d may encircle hole 560.

FIG. 16D is a plan view of a plate 500 with a plurality of slots 542a, 542b, . . . placed in a spiral pattern according to an embodiment. The plurality of slots 542a, 542b, . . . may extend into a surface of the plate 500 and one or more of the plurality of slots 542a, 542b, . . . may extend through the entire depth of the plate 500. The plurality of slots 542a, 542b, . . . may encircle hole 560.

FIG. 16E is a sectional view of plate 500 along line A-A′ of FIG. 16A according to an embodiment. As illustrated in FIG. 16E, perforations 540a and 540b extend through the entire depth of the plate 500, thereby creating holes in the plate 500.

FIG. 16F is a sectional view of plate 500 according to another embodiment. As illustrated in FIG. 16F, rather than providing perforations into the plate 500, protuberances 530a to 530d are provided that extend from the plate 500. As illustrated, protuberances 530a and 530b are provided on a lower side of the plate 500, and protuberances 530c and 530d are provided on an upper, opposite, side of the plate 500. Alternatively, protuberances 530 may be provided on only one side of the plate 500. The protuberances 530 may be provided in pin form, removable from a surface of the plate 500. Also, the protuberances 530 may form one or more of a textured surface and pattern on a surface of the plate 530. The protuberances may be formed of photocatalyst coating.

FIG. 16G is a perspective view of plate 500 illustrating alternating rows of protuberances 530 and perforations 540. Variation of patterns of one or both of the rows of protuberances 530 and perforations 540 on the plate 500 allows for inducing off axis rolling vortices in the airflow, as illustrated in FIG. 13C.

Therefore, the present disclosure provides an air purification device that includes an inlet; a first chamber including at least one first emitter and at least one filter media, wherein the at least one first emitter is configured to irradiate at least a surface of the at least one filter media; a second chamber configured to receive air from the first chamber, wherein the second chamber includes at least one second emitter; and at least one plate provided in the second chamber, wherein the at least one second emitter is configured to irradiate at least a portion of the at least one plate.

The present disclosure also provides an air purification device that includes an inlet, a first chamber, a second chamber configured to receive air from the first chamber, and at least one plate provided in the second chamber, wherein the second chamber includes at least one emitter configured to irradiate at least a portion of the at least one plate, and wherein the second chamber is configured to induce cyclonic air flow therein.

The present disclosure also provides an air purification device that includes an inlet, a chamber configured to induce cyclonic flow of air from the inlet, and at least one plate provided in the chamber, wherein the chamber includes at least one emitter configured to irradiate at least a portion of the at least one plate.

The present disclosure additionally provides a method of purifying air by an air purification device that includes an inlet, a first chamber, a second chamber including at least one plate and at least one emitter, and an exhaust, with the method including flowing air from the inlet through the first chamber and the second chamber to the exhaust; and irradiating, by the at least one emitter, at least a portion of the at least one plate, wherein the second chamber is configured to induce cyclonic air flow therein.

The devices and methods disclosed herein provide increased flow rate, e.g., 55-65 CFM, while maintaining residence time and homogeneous flow, thus minimizing dead zones and maximizing efficiency. The devices and methods disclosed herein also provide an expanded photocatalyst reaction area that minimizes UV-C light leakage. In practice, the disclosed devices and methods may be used to remove air contaminates susceptible to photocatalytic oxidation and/or the UV-A filter. For instance, the device and method could be used in medical settings, industrial settings, agricultural settings, food production, transportation, and related settings in which control air contaminates is desired.

Reference numbers

| | |
|---|---|
| 1000 | air purification device |
| 100 | inlet |
| 100a, 100b | inlet fan |
| 200 | first chamber |
| 220 | first emitter |
| 250 | filter media |
| 400 | second chamber |
| 410 | first side |
| 420 | second side |
| 430 | circular wall |
| 440 | internal fan |
| 500 | plate |
| 530 | protuberance |
| 535 | plate photocatalyst coating |
| 540 | perforation |
| 542 | slot |
| 544 | triangular cutout |
| 560 | hole |
| 600 | second emitter |
| 900 | exhaust |
| 920 | outlet tube |
| 935 | tube photocatalyst coating |
| 940 | cap |

In referring to the description, specific details are set forth in order to provide a thorough understanding of the examples disclosed. In other instances, well-known methods, procedures, components, and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans may employ such variations as appropriate, for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. An air purification device, comprising:
an inlet;
a first chamber including at least one first emitter and at least one filter media, wherein the at least one first emitter is configured to irradiate at least a surface of the at least one filter media;
a second chamber configured to receive air from the first chamber, wherein the second chamber includes at least one second emitter; and
at least one plate provided in the second chamber, wherein
the at least one second emitter is configured to irradiate at least a portion of the at least one plate, and
the second chamber is configured to induce cyclonic air flow therein.

2. The air purification device of claim 1, wherein
at least a part of a surface of the at least one filter media is coated with a photocatalyst configured to be activated by illumination from the at least one second emitter to cause photocatalytic oxidation of at least one of a particle and a gaseous molecule flowing through the air purification device.

3. The air purification device of claim 1, wherein
the second chamber includes a first side and a second side,
the at least one plate is offset from the first side and the second side,
the at least one second emitter is fixed on the first side, and
a substantially circular wall connects the first side and the second side.

4. The air purification device of claim 1, wherein
air enters the second chamber in a direction orthogonal to a direction of air exiting through an exhaust of the second chamber,
the exhaust is positioned substantially in a center of the second chamber, and
the exhaust is formed as an outlet tube.

5. The air purification device of claim 4, wherein
the outlet tube extends through a hole substantially in a center of the at least one plate.

6. The air purification device of claim 4, wherein
at least a part of the outlet tube is coated with a photocatalyst configured to be activated by illumination from the at least one second emitter.

7. The air purification device of claim 4, wherein
at least a part of a surface of the at least one of the at least one plate and the outlet tube are coated with a photocatalyst configured to be activated by illumination from the at least one second emitter to cause photocatalytic oxidation of at least one of a particle and a gaseous molecule flowing through the air purification device.

8. The air purification device of claim 1, wherein
the at least one plate includes at least one of a single perforation or a plurality of perforations,
air flowing through to the second chamber flows at least one of into and/or through the at least one of the single perforation or the plurality of perforations, and
the plurality of perforations are at least one of:
elongated in shape,
substantially circular in shape,
substantially square in shape,
substantially triangular in shape,
substantially rectangular in shape,
arranged along a substantially flat surface,
arranged as a helix, and
arranged as a spiral.

9. The air purification device of claim 1, further comprising:
a cap positioned adjacent to an exhaust of the second chamber, wherein
the cap is configured to reduce emission of radiation from the outlet, and at least one of:
the cap has a substantially flat surface positioned adjacent to an outer circumference of a distal end of the exhaust;
the cap has a substantially triangular surface positioned adjacent to an outer circumference of a distal end of the exhaust;
an internal circumference of the cap is tapered;
a distal end of the cap is conically shaped;
an interior circumference of a proximal end of the cap is wider than an interior circumference of the distal end of the cap, with the distal end extending towards the second chamber; and
an interior circumference of a proximal end of the cap is narrower than an interior circumference of a distal end of the cap, with the distal end extending away from the second chamber.

10. The air purification device of claim 1, further comprising:
at least two fans positioned adjacent to the inlet, wherein
the at least two fans have an airflow channel aspect ratio configured to provide homogenous airflow through the first chamber.

11. The air purification device of claim 1, wherein
the at least one second emitter is arranged in a circular configuration corresponding to a shape of the at least one plate.

12. The air purification device of claim 1, wherein
the at least one second emitter is arranged as a plurality of strips, and
each strip of the plurality of strips is arranged in a shape corresponding to a shape of the at least one plate.

13. An air purification device, comprising:
an inlet;
a first chamber;
a second chamber configured to receive air from the first chamber; and
at least one plate provided in the second chamber,
wherein the second chamber includes at least one emitter configured to irradiate at least a portion of the at least one plate, and
wherein the second chamber is configured to induce cyclonic air flow therein.

14. The air purification device of claim 13, wherein
wherein the at least one plate is substantially circular.

15. The air purification device of claim 13, wherein
the at least one plate is positioned apart from each wall of the second chamber.

16. The air purification device of claim 13, wherein
the at least one plate includes a plurality of perforations,
at least one perforation of the plurality of perforations extends through the at least one plate, and
air flowing through the second chamber flows into the at least one perforation of the plurality of perforations.

17. The air purification device of claim 13, wherein
the at least one plate includes a plurality of protuberances, and
the plurality of protuberances form vortexes in air flowing through the second chamber.

18. An air purification device, comprising:
an inlet;
a chamber configured to induce cyclonic flow of air from the inlet; and
at least one plate provided in the chamber, wherein
the chamber includes at least one emitter configured to irradiate at least a portion of the at least one plate.

19. The air purification device of claim 18, wherein
the at least one plate includes a plurality of perforations, with at least one perforation of the plurality of perforations extending through the at least one plate, and
air flowing through to the chamber flows into or through the plurality of perforations.

20. The air purification device of claim 18, wherein
the at least one plate includes a plurality of protuberances, and
the plurality of protuberances form vortexes in air flowing through the second chamber.
